(19) United States
(12) Patent Application Publication
(10) Pub. No.: US 2009/0203998 A1
(43) Pub. Date: Aug. 13, 2009

(54) HEART RATE COUNTER, PORTABLE APPARATUS, METHOD, AND COMPUTER PROGRAM FOR HEART RATE COUNTING

(76) Inventors: Gunnar KLINGHULT, Lund (SE); Tomas Wassingbo, Lund (SE)

Correspondence Address: WARREN A. SKLAR (SOER);RENNER, OTTO, BOISSELLE & SKLAR, LLP, 1621 EUCLID AVENUE, 19TH FLOOR, CLEVELAND, OH, 44115, US

(21) Appl. No.: 12/030,440

(22) Filed: Feb. 13, 2008

(51) Int. Cl. A61N 5/06; A61N 1/00; A61B 8/00

(52) U.S. Cl. 600/443; 607/88; 607/72

(57) ABSTRACT

A heart rate counter comprising a light source; a camera arranged in a vicinity if the light source such that light from the light source is able to be registered through living tissue, and arranged to acquire a series of images of the light transmitted through the tissue; and an image processor connected to the camera and arranged to determine a heart rate by photoplethysmography from the series of images is disclosed. Further, corresponding portable apparatus, computer program, and method are disclosed

### FIELD OF INVENTION

The present invention relates to a heart rate counter, a portable apparatus with heart rate counting features, a method for heart rate counting, and computer program for heart rate counting.

### BACKGROUND

Heart rate monitors are widely used by top-level sportsmen for enhancement of practising, but have become more common also among people having a particular interest in keeping fit.

An example of a heart rate monitor is disclosed in GB 2 409 040 A. The heart rate monitor comprises a chest belt with a heart rate sensor, which provides signals to a processor that converts the signals to information available to the user. A particular feature of this heart rate monitor is that it is able to provide an audio output of the information. It is further suggested that the heart rate monitor be combined with a radio, music player, or watch.

Heart rate monitors are normally specialized gear that is still too costly or pretentious for e.g. the ordinary jogger. Therefore, there is a demand for a heart rate monitor that could be available also to persons that do not belong to the group of top-level sportsmen or people addicted to fitness culture.

### SUMMARY

The present invention is based on the understanding that exercising and a common interest in fitness is widely spread, as well as portable apparatuses, such as mobile phones, which are commonly carried also during exercising may be used for aid in the exercising. The inventors have realized that image capturing and image processing can determine heart rate, which is of common interest during exercising, and that image capturing and image processing are functions that are commonly available in the portable apparatuses in question. The heart rate can be determined by plethysmography, i.e. by observing volume changes of organs, by optical means, i.e. photoplethysmography. By observing e.g. blood vessels in this way, heart rate can be determined. The inventors have further realized that implementation can be provided neatly by an application in the portable apparatus, which in turn can be implemented by software. Thus, no or few extra hardware components may be needed.

Further, on the other hand, the inventors have found that besides being able to provide a heart rate counter in apparatuses that common people are used to, the present invention may also be beneficial to persons that do belong to the group of top-level sportsmen or people addicted to fitness culture since the software abilities can further enhance functions of heart rate counters that this group may demand.

According to a first aspect of the present invention, there is provided a heart rate counter comprising a light source; a camera arranged in a vicinity if the light source such that light from the light source is able to be registered through living tissue, and arranged to acquire a series of images of the light transmitted through the tissue; and an image processor connected to the camera and arranged to determine a heart rate by photoplethysmography from the series of images.

In this context, the term “camera” should be construed as any image capturing means, as will be understood from the detailed description, which besides use for heart rate registration may be intended for other image capturing.

The heart rate counter may further comprise a display arranged to display a result of heart rate counting. The result may comprise a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof.

The camera may further be arranged for general image capturing. The image processor may be arranged to discriminate images of living tissue from which heart rate is determinable from general images such that a heart rate counting is initiated.

According to a second aspect of the present invention, there is provided a mobile portable apparatus comprising a heart rate counting application; and means for heart rate counting according to the first aspect of the present invention arranged to support the heart rate counting application when the heart rate counting application is running.

The portable apparatus may further comprise a display arranged to display a result of heart rate counting. The portable apparatus may be arranged to provide the result, which may comprise a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof.

The camera may further be arranged for general image capturing, e.g. being the ordinary camera of the portable apparatus. Similarly, the light source may be the ordinary light source that is used for general image capturing.

The image processor may be arranged to discriminate images of living tissue from which heart rate is determinable from general images such that the heart rate counting application is initiated. Thus, the heart rate counting application may be automatically started when images are determined to be captured of a body tissue from which the pulse can be determined.

The portable apparatus may be a mobile phone, a portable digital assistant, a digital camera, a media player, etc.

According to a third aspect of the present invention, there is provided a method for heart rate counting comprising illuminating living tissue by a light source of a portable apparatus; acquiring a series of images of the light transmitted through the tissue by a camera of the portable apparatus; and processing the series of images to determine a heart rate by photoplethysmography from the series of images by an image processor of the portable apparatus.

The method may further comprise displaying a result of heart rate counting on a display of the portable apparatus. The result may comprise a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof. Thus, the method may comprise providing any of these results.

The method may further comprise discriminating images of living tissue from which heart rate is determinable from general images by the image processor; and initiating a heart rate counting application of the portable apparatus if living tissue from which heart rate is derivable is determined in the series of images.

According to a fourth aspect of the present invention, there is provided a computer readable medium comprising program code, which when executed by a processor is arranged to cause the processor to perform a heart rate counting application comprising performing illumination of living tissue by a light source; acquisition of a series of images of the light transmitted through the tissue by a camera; and processing of the series of images to determine a heart rate by photoplethysmography from the series of images.

The program code may further cause the processor to perform displaying a result of heart rate counting on a display. The program code may further cause the processor to perform provision of a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof, to be provided as the result.

The program code further cause the processor to perform discrimination of images of living tissue from which heart rate is determinable from general images by the image processor; and initiation of the heart rate counting application of the portable apparatus if living tissue from which heart rate is derivable is determined in the series of images.

According to a fifth aspect of the present invention, there is provided a computer program comprising instructions, which when executed by a processor are arranged to cause the processor to perform the method according to the third aspect of the present invention.

### BRIEF DESCRIPTION OF THE DRAWINGS

FIG. 1 is a block diagram schematically illustrating a heart rate counter.

FIG. 2 illustrates a computer program product according to an embodiment of the present invention.

FIGS. 3a and 3b illustrate an exemplary portable apparatus according to an embodiment of the present invention.

FIGS. 4a and 4b illustrate an exemplary portable apparatus according to an embodiment of the present invention.

FIG. 5 illustrates use of an apparatus for heart rate counting according to an embodiment of the present invention.

FIG. 6 illustrates use of an apparatus for heart rate counting according to an embodiment of the present invention.

FIG. 7 is a flow chart illustrating a method according to an embodiment of the present invention.

FIGS. 8a and 8b schematically illustrates top and side views, respectively, of a part of a display, and FIG. 8c schematically illustrates a variant of operation to the one illustrated in FIG. 8b.

FIG. 9 illustrates an exemplary portable apparatus in use for photoplethysmographic measurements.

### DETAILED DESCRIPTION

The present invention is based on photoplethysmography, alternatively on pulse oximetry, which both are based on light reflection/absorption in blood hemoglobin which changes by the pulsation of blood through the blood vessels. Thus, by illuminating body tissue by a light source, the reflected light can be observed, and based on changes in reflection in certain wavelengths, the pulse can be calculated. To discriminate the desired changes in light acquired by an image acquisition means, e.g. a camera imaging the illuminated body tissue, from other variations caused by for example changes in distance between the body tissue and the light source/camera, or changes in ambient light, proper signal processing is performed by an image processor. The image processor can discriminate the desired changes in light since the actual wavelengths are known, and the heart rate is within a rather limited rate. Thus, a rather simple and reliable calculation is possible. With a bit more complex calculation, other parameters, such as oxygenation, can be derived.

As indicated above, detection of the reflected light is performed by an image acquisition means such as a camera. To just detect the changes in reflection, a photo sensor would be sufficient, but the present invention is based on the insight that apparatuses primarily used for other purposes and having a camera and image processing means, e.g. digital camera, media player, or mobile phone, can be used for heart rate monitoring with addition of no or few hardware elements. For example, a media player used for playing music during exercise having features according to any of the presented embodiments can be further arranged to select music based on the determined pulse rate. This selection can also be based on an aggregate of pulse rate, and any of accelerometer outputs of the device, positioning information, measured body temperature, and/or information from a predetermined exercise program. Another example can be where call handling in a mobile phone is based on pulse rate, where phone calls are rejected or re-directed if the pulse rate is above a determined pulse rate threshold, e.g. 100 heartbeats per minute. Another example is to control illumination effects of the device based on the determined pulse rate, such as color, modulation, or intensity. The pulse rate can be stored in the device, or if the device has communication capabilities, as for the mobile phone case, the pulse rate can be transmitted and be made available at a remote location, e.g. to a coach.

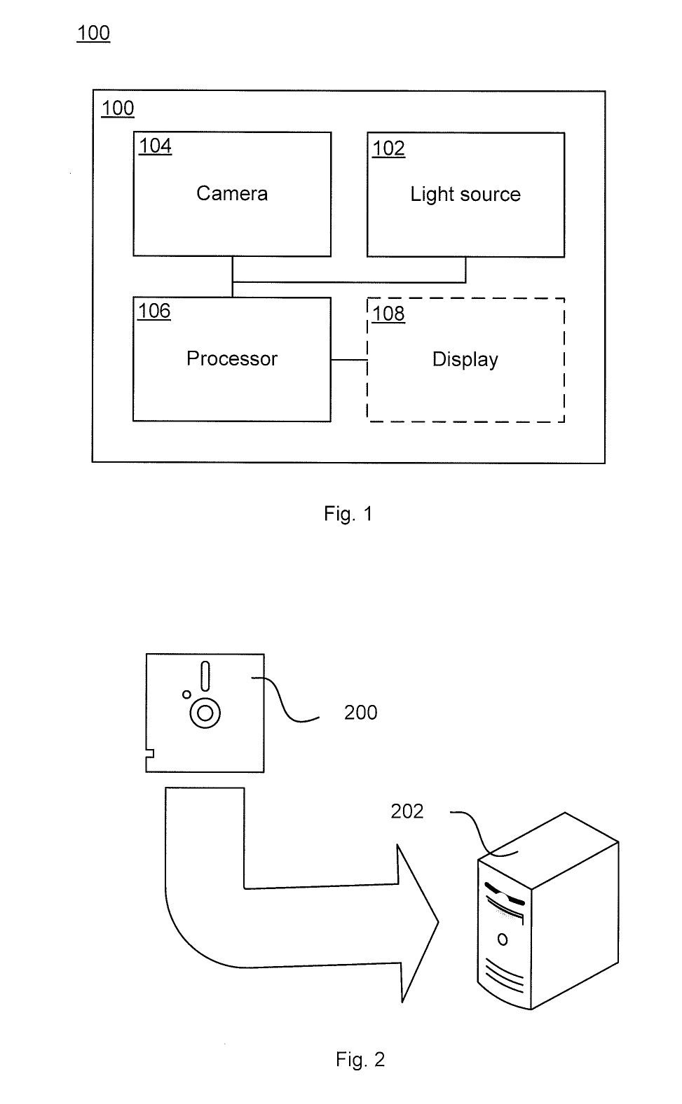

FIG. 1 is a block diagram schematically illustrating a heart rate counter 100 according to an embodiment of the present invention. The term “heart rate counter” should be construed functionally, i.e. as an application within an apparatus having also other abilities and using selected elements of the apparatus for implementing the heart rate counter 100, which elements are described below as “comprised” in the heart rate counter 100. The heart rate counter 100 comprises a light source 102 which is arranged to illuminate body tissue of a user whose heart rate is to be determined. According to what has been demonstrated above about photoplethysmography and pulse oximetry, reflected light from the body tissue holds information on blood flow, and also other parameters related to the blood. From this, the heart rate is determinable. This is performed by a camera 104, which acquires a series of images of the body tissue, e.g. a finger, a part of a palm of a hand, an ear lobe, a forehead, or other part where blood vessels are “optically” available. The series of images is processed by a processor 106, e.g. an image processor or signal processor arranged to process the large amount of data comprised in the images to provide the desired data on the blood flow, e.g. heart rate and/or oxygenation. The processor 106 can also be a general processor of the heart rate counter, or be a combination of processors co-operating. Optionally, the result of the determined data is presented on a display 108. Other alternatives, or complements, to presenting the result are tactile or audio presentation, e.g. a speaking voice that is generated or indication beeps, by means (not shown) arranged for this, such as a vibrator or a speaker. For example, upper and lower thresholds may be set by the user, and an audible or tactile indication is given to the user if the heart rate is out of the range defined by the thresholds.

As discussed above, the heart rate counter is implementable with no or few hardware additions in an apparatus that normally comprises a light source, a camera, and a processor. The heart rate counter is implemented by adding a heart rate counter application in form of software. Thus, the heart rate counter is suitable for implementation with aid of processing means, such as general, signal, and/or image processors. The computer program preferably comprises program code, as illustrated in FIG. 2, which is stored on a computer readable medium 200, which can be loaded and executed by a processing means 202 to cause it to perform a heart rate counting application comprising performing illumination of living tissue by a light source, acquisition of a series of images of the light transmitted through the tissue by a camera, and processing of the series of images to determine a heart rate by photoplethysmography from the series of images. The program code can further cause the processing means to perform displaying a result of heart rate counting on a display controlled by the processing means. The program code can further cause the processor to perform provision of a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof, to be provided as the result. The program code can also be arranged to determine other parameters from the reflected light, such as oxygenation. The amount derived from the heart rate and parameters specific to the user can be calculated energy consumed during exercise. The amount derived from the heart rate and information from another application, which can be positioning information from a positioning application of the apparatus, e.g. a GPS application, or altitude information from an altimeter application, can be an indication on performance in relation to the physical effort of the user. The animation can for example be a diagram illustrating heart rate to other parameters, such as speed, altitude or time, or an animation looking like an electrocardiogram. The processing means 202 and computer program product 200 can be arranged to execute the program code sequentially where actions are performed stepwise, but mostly be arranged to execute the program code on a real-time basis where actions are performed upon need and availability of data. The processing means 202 is preferably what normally is referred to as an embedded system. Thus, the depicted computer readable medium 200 and processing means 202 in FIG. 2 should be construed to be for illustrative purposes only to provide understanding of the principle, and not to be construed as any direct illustration of the elements.

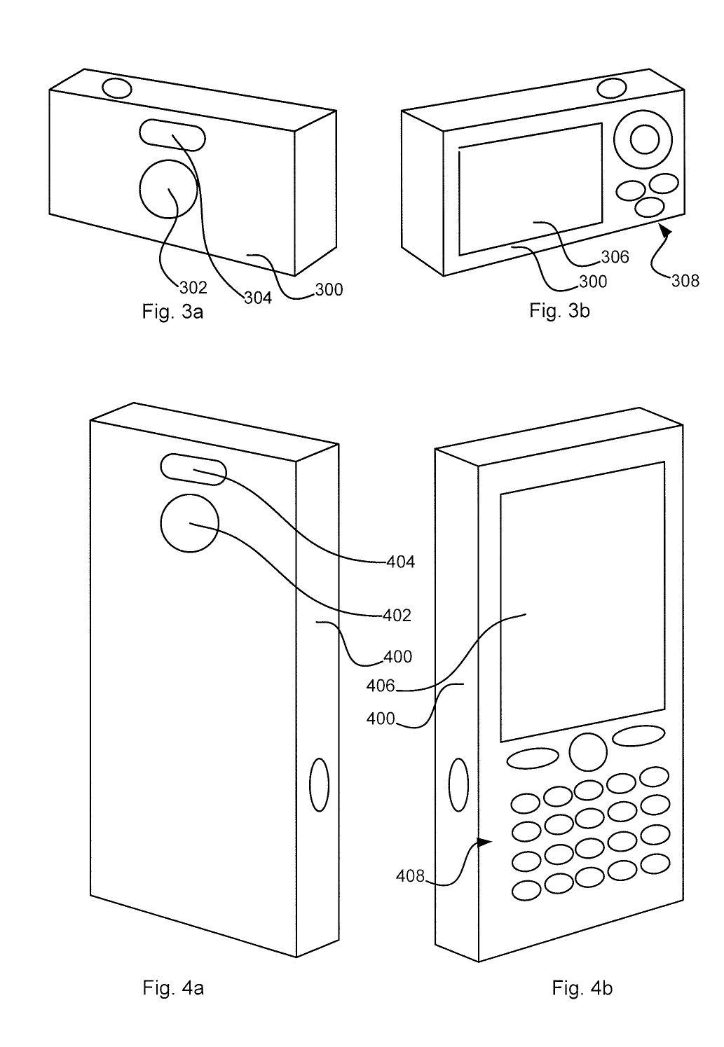

FIGS. 3a and 3b illustrate an exemplary portable apparatus being a digital camera 300 having a heart rate counter according to an embodiment of the present invention. The digital camera 300 comprises a lens 302 arranged close to a light source 304 such that body tissue can be illuminated and imagined when placed in front of the lens 302 and the light source 304. As described above, heart rate and other parameters associated with blood flow can thus be determined by the apparatus. The result can be presented on a display 306, which type of result can be chosen by means of input means 308 of a user interface. Besides this, the digital camera 300 can of course be used as a normal digital camera. A processing means of the digital camera can be arranged to detect that the acquired images are of a body tissue reflecting light from which heart rate etc. can be determined and then automatically start the heart rate application.

FIGS. 4a and 4b illustrate an exemplary portable apparatus being a communication apparatus or a personal digital assistant, or both, 400 having a heart rate counter according to an embodiment of the present invention. The apparatus 400 comprises a camera 402 arranged close to a light source 404, e.g. a light emitting diode (LED), an organic LED (OLED), an xenon flash, etc., such that body tissue can be illuminated and imagined when placed in front of the camera 402 and the light source 404.

An example setup can be that the body tissue is illuminated by a backlit display of a mobile phone, where the camera 402 is a camera arranged for video call, which is normally arranged in vicinity of the display.

Another example is that the body tissue is illuminated by means of the display, which has the features of having a display also arranged for image capturing, i.e. image sensors be embedded in the display device. In such case, the display itself will comprise both the camera 402 and the light source 404. Such a display is described e.g. in press release of 25 May 2004, Seattle, by Toshiba America Electronics named “TOSHIBA DEBUTS FIRST FULL-COLOR ‘SYSTEM ON GLASS’ (SOG) INPUT DISPLAY WITH IMAGE CAPTURE TECHNOLOGY—New Color LCD Prototype, which Captures High-Resolution Digital Content via Embedded Sensors, to be Demonstrated at SID 2004 in Seattle”.

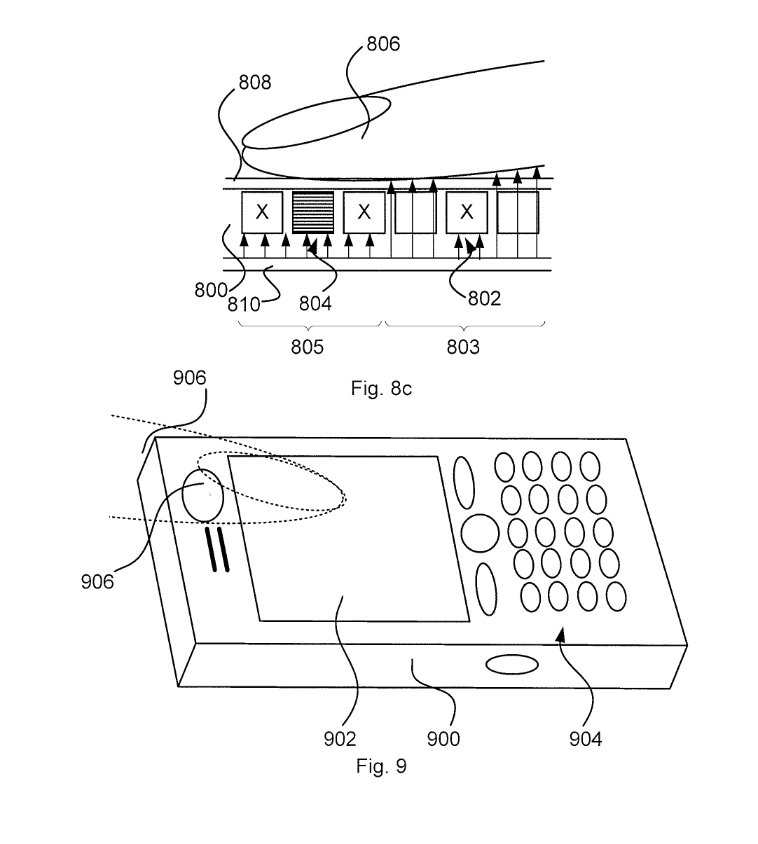

FIGS. 8a and 8b schematically illustrates top and side views, respectively, of a part of a display 800 with integrated image acquisition means, i.e. a “camera” within the meaning of this disclosure. The normal display function is provided by means of the picture elements (pixels) 802, while the embedded light sensitive pixels 804 (indicated by an “x”) are adapted to acquire an image of an object placed on the display 800. A popular application is to acquire an image of a business card placed on the screen for storing the information in e.g. a phone book of the device having the display 800. In the embodiment of the present invention, the image acquiring display 800 is used for registering images of a body tissue 806 to determine e.g. pulse rate, as illustrated in FIG. 8b where the body tissue is put on a display cover 808 covering the display 800. For providing the light source, backlight 810 of the display 800 is used to provide light (straight arrowed lines).

FIG. 8c illustrates a variant of the approach demonstrated with reference to FIG. 8b. In FIG. 8c, the pixels 802 are functionally divided into a first area 803 arranged for illumination of the body tissue 806, and a second area 805 for image capturing. An advantage of this is that interference between provided illumination and reflected light can be avoided, which can improve sensitivity. The position of the first area 803 can be determined from image analysis, whereby image pixels of the second area 805 are made “dark”, i.e. put into a low light transmission state, and image pixels of the first area 803 are made “bright”, i.e. put into a high light transmission state. Preferably, pixels that are not part of the first and second areas 803, 805, i.e. pixels at the rest of the screen not used for the photoplethysmographic measurement, can be used for displaying any information, or be put into a “dark” state.

It should be noted that FIGS. 8a to 8c are not drawn into scale, but are drawn to schematically illustrate the principles on which embodiments of the present invention can rely. Normally, the pixels 802, 804 are smaller and more numerous in relation to the size of the illustrated body tissue 806.

FIG. 9 illustrates an exemplary portable apparatus, a mobile phone 900 having a display 902, a user input 904, and a camera 906, that is normally used for video telephony and thus directed to an expected position of a user, i.e. in a direction substantially normal to the display 902. Upon use according to embodiments of the present invention, light is provided by the display 902, which usually is backlit, to a body tissue 908, e.g. the user's finger placed over a part of the display 902 and the camera 906. The display 902 can be provided with an extra light source, or the existing light source can be arranged for providing amplified light for backlighting in the area close to the camera 906. The extra light source or the amplification of backlight can be initiated together with a heart rate application of the mobile phone. Automatic start of the heart rate application can be provided, as will be further described below. With aid of any of these features, photoplethysmographic measurements as demonstrated herein can be performed by the mobile phone 900, which measurements can give results in heart rate or oxygenation, which results can be used for calculating further information in different applications of the mobile phone 900.

Returning to FIG. 4, it should be noted that the light source 404 can work with light that is invisible to human, such as infrared light, since many cameras are suitable for registering also infrared light. The actual area of the camera 402 and the light source 404 where the body tissue is to be placed can have a touch sensitive input (not shown), such that the pulse rate application can be automatically started when a proper input is registered at the touch sensitive input. Automatic start of the pulse rate application can also be when image processing recognizes a captured image with certain characteristics emanating from illuminated body tissue close to the camera 402.

As described above, heart rate and other parameters associated with blood flow can thus be determined by the apparatus 400. The result can be presented on a display 406, which type of result can be chosen by means of input means 408 of a user interface, such as a keypad. Besides this, the apparatus 400 can of course be used for normal operation of a mobile phone or personal digital assistant, such as calling, using calendar applications, web browsing, etc. A processing means of the apparatus 400 can be arranged to detect that the acquired images are of a body tissue reflecting light from which heart rate etc. can be determined and then automatically start the heart rate application.

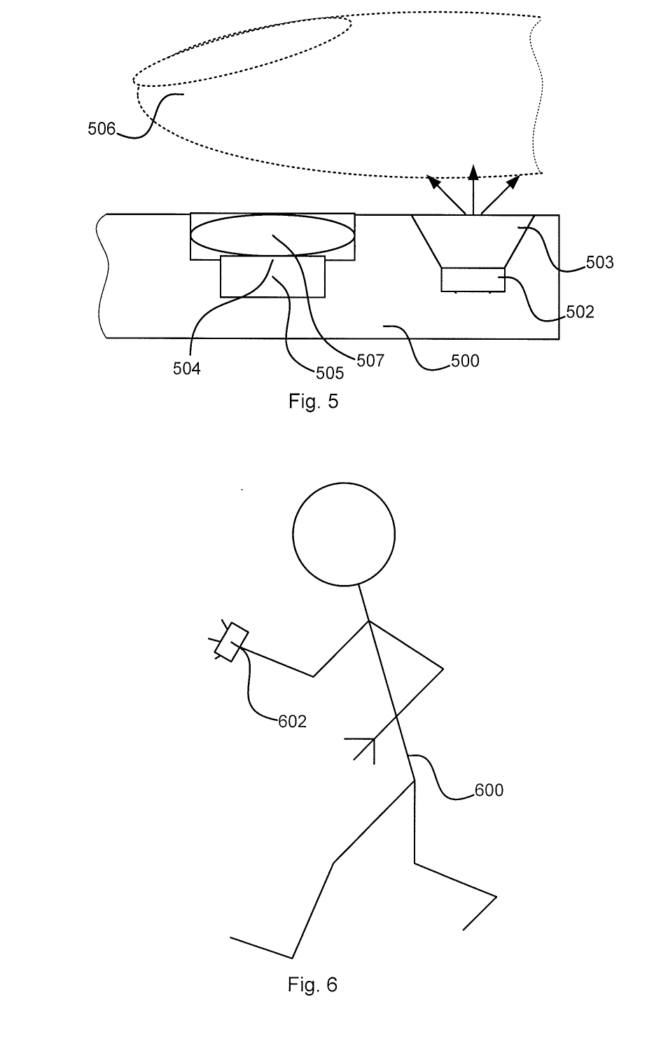

FIG. 5 illustrates exemplary use of an apparatus 500 for heart rate counting according to an embodiment of the present invention. The apparatus 500 comprises a light source 502 and a camera 504 arranged close to each other such that a piece of body tissue 506, e.g. a finger or a palm of a hand, can be placed over the light source 502 and the camera 504. The light source 502 can be arranged with means 503 for providing a desired light distribution, such as a reflector or a lens. The camera 504 can comprise an image sensor 505 registering an image provided through a lens 507. Light 508 from the light source 502 is reflected by haemoglobin of the blood flowing in blood vessels in the body tissue 506, and the reflected light can be observed by the camera 504. Based on changes in reflection in certain wavelengths, the pulse can be calculated. To discriminate the desired changes in light acquired by the camera imaging the illuminated body tissue, from other variations caused by for example changes in distance between the body tissue and the light source/camera, or changes in ambient light, proper signal processing is performed by an image processor. The image processor can discriminate the desired changes in light since the actual wavelengths are known, and the heart rate is within a rather limited rate. Thus, a rather simple and reliable calculation is possible. With a bit more complex calculation, other parameters, such as oxygenation, can be derived.

FIG. 6 illustrates exemplary use of an apparatus for heart rate counting according to an embodiment of the present invention. Consider a user 600 performing physical exercise, e.g. jogging, and bringing her mobile portable apparatus 602, e.g. a digital camera, media player, mobile phone, or personal digital assistant, having image acquisition features. The apparatus 602 is held in her hand with its light source and camera facing her palm of the hand while jogging. She will then be able to register heart rate or other parameters as described above. The result of the registered data can be shown to her while jogging, and/or be stored such that she is able to check any desired data afterwards, e.g. for keeping a training diary.

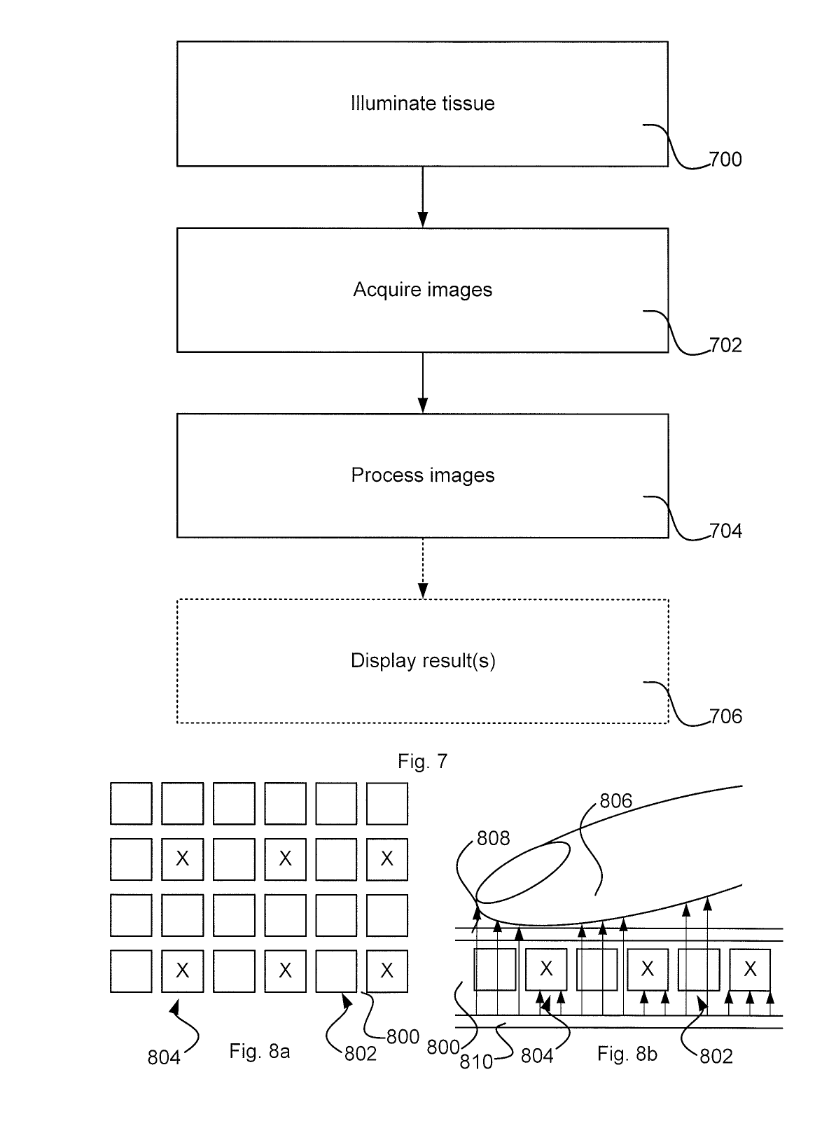

FIG. 7 is a flow chart illustrating a method according to an embodiment of the present invention. In a body tissue illumination step 700, a living tissue having the blood flow on which the heart rate is to be determined is illuminated by a light source of a portable apparatus. As the light is absorbed and reflected by hemoglobin of the blood flowing in blood vessels in the body tissue as described above, a series of images is acquired in an image acquisition step 702 of the illuminated body tissue, and thus the reflected light which holds information on at least blood flow, and thus heart rate. As discussed above, other parameters can also be derived, such as oxygenation. This information is gained in an image processing step 704 where the series of images is processed to provide heart rate, and possibly other parameters as well. The heart rate is preferably derived by photoplethysmography. The derived parameter or parameters from the series of images, and possibly together with information from other applications, such as positioning, altitude, or temperature measurements, and also possibly together with user specific parameters, a set of results is provided, wherein the set of results at least comprises the heart rate. The set of results can further comprise any amount derivable from the heart rate and user specific parameters, any amount derived from the heart rate and information from another application, an animation indicating the heart rate, a feedback indication indicating acquisition quality, and/or the series of acquired images. The set of results is optionally displayed in a result display step 706. Alternatives, or complements to the display step 706 can be storing the results such that the user is able to check any desired data afterwards, e.g. for keeping a training diary, and/or presenting results by tactile and/or audible output. Optionally, the steps 700-708 can be initiated upon acquisition of images where images of living tissue from which blood flow, and thus heart rate, is determinable are discriminated from general images captured, e.g. normal photos of flowers, views, etc. Based on this, if living tissue as described above is determined to be imaged, the steps 700-708 are initiated. Thus, an “automatic” start of the heart rate application is provided.

An additional feature for “automatic” start of the heart rate application can be implemented by an accelerometer in the portable apparatus which can detect if the person carrying the apparatus is walking or running, i.e. perform a so called pedometer function. The detection of walking or running together with determined images of living tissue from which blood flow and thus heart rate is determinable can be used as a trigger for starting the heart rate application. Where the display is used as light source, and a touch sensitive display is used, a detection of a long touch of the display, e.g. in an area where heart rate measurements are possible (cf. FIG. 8 or 9), can be used as a trigger for starting the heart rate application. Any of the demonstrated triggers can be used in combination to gain a reliable starting of the heart rate application.

1. A heart rate counter comprising
a light source;
a camera arranged in a vicinity if the light source such that light from the light source is able to be registered through living tissue, and arranged to acquire a series of images of the light transmitted through the tissue; and
an image processor connected to the camera and arranged to determine a heart rate by photoplethysmography from the series of images.

2. The heart rate counter according to claim 1, further comprising a display arranged to display a result of heart rate counting.

3. The heart rate counter according to claim 2, wherein the result comprises a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof.

4. The heart rate counter according to claim 1, wherein the camera is further arranged for general image capturing.

5. The heart rate counter according to claim 4, wherein the image processor is arranged to discriminate images of living tissue from which heart rate is determinable from general images such that a heart rate counting is initiated.

6. A mobile portable apparatus comprising
a heart rate counting application;
a light source;
a camera arranged in a vicinity if the light source such that light from the light source is able to be registered through living tissue, and arranged to acquire a series of images of the light transmitted through the tissue; and
an image processor connected to the image acquisition means and arranged to determine a heart rate by photoplethysmography from the series of images when said heart rate counting application is running.

7. The apparatus according to claim 6, further comprising a display arranged to display a result of heart rate counting.

8. The apparatus according to claim 7, wherein the result comprises a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof.

9. The apparatus according to claim 6, wherein the camera is further arranged for general image capturing.

10. The apparatus according to claim 9, wherein the image processor is arranged to discriminate images of living tissue from which heart rate is determinable from general images such that the heart rate counting application is initiated.

11. A method for heart rate counting comprising
illuminating living tissue by a light source of a portable apparatus;
acquiring a series of images of the light transmitted through the tissue by a camera of the portable apparatus; and
processing the series of images to determine a heart rate by photoplethysmography from the series of images by an image processor of the portable apparatus.

12. The method according to claim 11, further comprising displaying a result of heart rate counting on a display of the portable apparatus.

13. The method according to claim 12, wherein the result comprises a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof.

14. The method according to claim 11, further comprising
discriminating images of living tissue from which heart rate is determinable from general images by the image processor; and
initiating a heart rate counting application of the portable apparatus if living tissue from which heart rate is derivable is determined in the series of images.

15. A computer readable medium comprising program code, which when executed by a processor is arranged to cause the processor to perform a heart rate counting application comprising performing
illumination of living tissue by a light source;
acquisition of a series of images of the light transmitted through the tissue by a camera; and
processing of the series of images to determine a heart rate by photoplethysmography from the series of images.

16. The computer readable medium according to claim 15, wherein the program code further causes the processor to perform displaying a result of heart rate counting on a display.

17. The computer readable medium according to claim 16, wherein the program code further causes the processor to perform provision of a heart rate value in beats per time unit, an amount derived from the heart rate and parameters specific to a user, an amount derived from the heart rate and information from another application, an animation indicating the heart rate, a feedback indication indicating acquisition quality, or the series of acquired images, or any combination thereof, to be provided as the result.

18. The computer readable medium according to claim 15, wherein the program code further causes the processor to perform
discrimination of images of living tissue from which heart rate is determinable from general images by the image processor; and
initiation of the heart rate counting application of the portable apparatus if living tissue from which heart rate is derivable is determined in the series of images.
